(19) United States
(12) Patent Application Publication
(10) Pub. No.: US 2025/0349425 A1
(43) Pub. Date: Nov. 13, 2025

(54) A SYSTEM AND METHOD FOR REQUESTING ADDITIONAL CLINICAL TESTS FOR A PATIENT

(71) Applicant: Olanike Adebayo, Dunmurry, Belfast (GB)

(72) Inventor: Olanike Adebayo, Dunmurry, Belfast (GB)

(21) Appl. No.: 18/871,967

(22) PCT Filed: May 25, 2023
(86) PCT No.: PCT/GB2023/051370

§ 371 Date: Dec. 5, 2024

(30) Foreign Application Priority Data
Jun. 6, 2022 (GB) 2208247.3

(51) Int. Cl. G16H 50/20; G16H 10/40; G16H 10/60

(52) U.S. Cl. CPC G16H 50/20; G16H 10/40; G16H 10/60

(57) ABSTRACT

A clinical test data management system and method, in particular for requesting additional clinical tests for a patient and escalating abnormal clinical test results, the method comprising obtaining identification data and clinical test result data, wherein the clinical test result data relates to a sample corresponding to the identification data; then determining whether the clinical test result data falls within a pre-determined range; and, based on the determination as to whether the clinical test result data falls within the pre-determined range, sending a signal to request an additional clinical test be performed on the sample corresponding to the identification data.

### FIELD OF THE INVENTION

The present disclosure relates to a clinical test data management system and method, in particular for requesting additional clinical tests for a patient and escalating abnormal clinical test results.

### BACKGROUND

In the past, medical professionals such as physicians, clinicians, nurses, and laboratory technicians used paper charts to track a patient's history and status. More recently, such professionals are increasingly using electronic medical records (EMR) and computer-based patient records (CPR), also known as electronic patient records (EPR), virtual patient records (VPR) and electronic health records (EHR), to leverage all available information to improve medical care.

These electronic records supplement or totally replace paper charts and records. Medical information systems (MIS) use EMR and CPR to maintain up-to-date, robust patient information while reducing the time, labour and other costs associated with managing such data. However, often data is manually input into EMR and CPR systems which can prove costly and time-consuming. Such systems may have hundreds or thousands of data fields per patient record, for example depending upon the physician's area of specialty.

Laboratory Information Management Systems (LIMS), also sometimes referred to as Laboratory Information Systems (LIS), are key to the functioning of a medical laboratory. LIMSs facilitate the keeping of electronic medical records (EMR) and computer-based patient records (CPR), as well as tracking the location, status, and upcoming tests due on biological samples, for example blood samples. In many cases, further tests are required following the results of an initial testing of a sample. For example, if a patient has a blood sample taken and that blood sample is tested for the presence of a specific pathogen, where the result is negative, further tests may be required to identify the presence of a different pathogen. Such additional tests may be known as add-on tests.

Each patient record may have a plurality of EMRs associated with it. EMRs may include many fields of data, including but not limited to sample type, date of sample, date of testing, type of testing, results of test, type of sample, test orders, when the results were disseminated to the relevant departments, location of sample, and more. For each sample this must be correctly cross-referenced with the correct patient record in order to record an accurate and comprehensive patient history.

In the event of an abnormal, concerning and/or urgent clinical test result, laboratory staff attempt to contact a relevant medical professional, such as a relevant physician or nurse, to alert them to the result. However, this process often results in several failed communication attempts, such as missed calls, due to the busy schedules of medical professionals. This can result in delays in reporting abnormal, concerning and/or urgent clinical test results and/or uncertainty as to whether a message communicating the clinical test result data has been received. In some cases, this may result in a delays to urgent medical treatment which can negatively affect a patient's medical care and outcomes.

### SUMMARY OF THE INVENTION

Aspects of the invention are as set out in the independent claims and optional features are set out in the dependent claims. Aspects of the invention may be provided in conjunction with each other and features of one aspect may be applied to other aspects.

In a first aspect there is provided a computer-implemented method for requesting additional clinical tests for a patient, the method comprising obtaining identification data and obtaining clinical test result data, wherein the clinical test result data relates to a sample corresponding to the identification data. The method then further comprises determining whether the clinical test result data falls above or below a predetermined threshold. In some examples, determining whether the clinical test result data falls above or below the predetermined threshold comprises determining whether the clinical test result data falls within a pre-determined range. Based on the determination as to whether the data falls above or below the predetermined threshold or within the pre-determined range, for example in the event that the clinical test result data does not fall within the pre-determined range, the method comprises sending a signal to request at least one additional clinical test be performed on the sample corresponding to the identification data. In some examples, the sample may be a blood sample from a patient; however, the skilled person will understand that any other types of medical sample may be used.

A limitation of existing record keeping systems, such as LIMSs, is that data must be input manually by medical professionals. In addition to being time consuming and labour intensive, this introduces room for error when data is input into the LIMS. EMR and CPR systems may contain hundreds or thousands of fields of data per patient record. Manual input can contain errors which may lead to unprocessed or lost samples, or test results being attributed to the wrong patient record. Further, the clinician in each department must go to the LIMS and manually check for specific test orders. An advantage of the present disclosure may be to streamline the process of requesting additional clinical tests. In particular, sending a signal to request an additional clinical test in response to an indication of the obtained clinical test result data may reduce time required for clinicians to manually check for specific test orders and request subsequent tests. Furthermore, the present disclosure may eliminate the requirement to obtain the identification data for a second time in order to request the additional test, reducing the time consuming and labour-intensive manual input of data, as well as reducing the likelihood of introducing error, for example by typographical error.

The present disclosure aims to improve upon communication between medical professionals in clinics, hospitals, and laboratories. The present invention may therefore relate to a system and method for communicating with one or more clinical data management systems and acting as an intermediary therebetween. More particularly, the system and method may be configured to request additional specific tests for a patient based on results from previous tests. This may be particularly relevant where such facilities are not in the same geographical location. For example, blood samples taken at several different clinics within a given area may all send their samples to a laboratory for testing via courier. It is therefore important that the samples are correctly labelled and identified by the various systems involved, despite each system potentially running different software, programming, operating systems and so on.

The present method may therefore increase efficiency of requesting additional clinical tests, not least by reducing clinician time required to check if clinical test result data has been received and request an additional clinical test. The method may also reduce the number of input errors which may ultimately result in fewer unprocessed or lost samples, or test results being attributed to the wrong patient record. The automated process may also facilitate maintaining patient confidentiality as the computer-implemented method may reduce the need for manual processing of confidential patient information.

In some examples, the identification (ID) data may comprise at least one of a sample ID, such as a sample number or label, or a patient ID, such as a patient number or identifier. The obtained identification data may correspond to identification data used by the hospital and/or clinicians, for example hospital ID. The identification data used by the hospital and/or clinicians may be the same or different to the identification data used by the laboratory. In some examples, obtaining the identification data further comprises obtaining the hospital identification data and identifying the laboratory identification data corresponding to the same patient and/or sample.

In some examples, the identification data is obtained from an API. In some examples, the API may obtain the identification data from a remote server, for example using cloud computing. In some examples, the method may be configured to directly obtain the identification data from the remote server. For example, laboratory identification data corresponding to the same patient and/or sample as the hospital identification may be identified and obtained from an API and/or remote server, for example from a database. This may be advantageous to reduce the number of input errors as a result of manual data input. This may ultimately result in fewer unprocessed or lost samples, or test results being attributed to the wrong patient record. The automated process may also facilitate maintaining patient confidentiality as the computer-implemented method may reduce the need for manual processing of confidential patient information.

In some examples, the method further comprises selecting at least one sample from a plurality of samples corresponding to the identification data for the additional clinical test to be performed on. Selecting the at least one sample from a plurality of samples may be based on the requirements of the clinical test and/or the properties of the samples, for example but not limited to, based on age or “freshness” of a sample, type of sample, sample integrity, whether a sample may have been contaminated, etc. This may be advantageous to select the most suitable sample for the additional clinical test requested, for example, some types of clinical tests may require that the sample tested is not older than 24, 36, or 48 hours old. However, the skilled person will understand that in other examples, the sample to be tested may be chosen from a plurality of samples by the clinician or laboratory staff.

In some examples, if no suitable sample exists, for example all samples corresponding to a patient and/or identification data are too old, an alert may be sent to a clinician to request that a suitable sample is taken from the patient. In some examples, a clinician may be prevented from requesting a specific type of additional clinical test if no suitable sample exists for the clinical test to be performed on. In this case, if a clinician attempts to request such a test, an alert may be sent to the clinician requesting a suitable sample be taken from the patient.

In some examples, sending the signal to the laboratory to request the additional clinical test be performed on the sample comprises sending the request to a laboratory information system (LIS) or laboratory information management system (LIMS), wherein the LIS or LIMS is then configured to send the request to the laboratory.

In some examples, sending the signal to request an additional clinical test be performed on the sample further comprises sending a signal to a sample storage facility to request that the sample to be tested be sent to a laboratory; and sending a signal to the laboratory to request the additional clinical test be performed on the sample. In other examples, sending the signal to the sample storage facility to request that the sample to be tested be sent to a laboratory; and sending the signal to the laboratory to request the additional clinical test be performed on the sample is performed by the LIS or LIMS.

The sample storage facility and/or laboratory may be on-site or off-site, for example wherein the laboratory may be a third-party laboratory. In some examples, the sample is retrieved robotically from the sample storage facility, based on the identification data. In some examples, the sample may be retrieved by correlating the identification data associated with a sample to a known location in the storage facility, for example position X in sample rack number Y. In some examples, the identity of the retrieved sample may be verified based on the identification data, for example by scanning an identification tag on the sample, such as a barcode, QR code, RFID tag, or other suitable means.

In some examples, after the requested test(s) have been performed by the laboratory, the sample may be retrieved robotically from the laboratory test equipment and returned to the sample storage facility. In some examples, the sample may be returned and stored in the sample storage facility by correlating the identification data associated with the sample to a known location in the storage facility, for example position X in sample rack number Y. In some examples, the storage location of the returned sample may be the sample as the location from which the sample was retrieved, however in other examples the sample may be returned to a different location and the known stored location of the sample is updated.

In some examples, the method may further comprise tracking the sample. Tracking the sample may comprise tracking the progress of the sample, for example tracking at least one of (i) the location of the sample; (ii) whether the sample has been retrieved from the sample storage facility; (iii) whether the sample has been sent to the laboratory; (iv) whether the sample has been received by the laboratory; (v) whether the sample has been tested; (vi) whether the sample has been returned to the sample storage facility; (vii) where the sample has been stored in the sample storage facility. This may be advantageous to reduce the number of unprocessed or lost samples.

In some examples, the method may comprise obtaining a request to perform a first clinical test on a sample, for example from a user. The method may then further comprise sending a signal to request a clinical test be performed on the sample corresponding to the identification data, in response to the request. The clinical test result data may then be subsequently obtained, wherein the clinical test result data relates to the first requested clinical test.

In some examples, the method further comprises receiving a request from a user for an additional clinical test to be performed on a sample, wherein the signal to request an additional clinical test be performed on the sample is sent in response to the request from the user. In some examples, the method further comprises sending a signal to suggest at least one type of recommended additional clinical test based on the determination as to whether the clinical test result data falls within a pre-determined range, for example in the event that the clinical test result data falls outside the pre-determined range, and receiving the request from the user for at least one additional clinical test to be performed on a sample in response to the suggestion. However, in other examples, the signal to request an additional clinical test is sent based on the previously obtained clinical test result data without user input, for example based on the determination that the clinical test result data falls outside a pre-determined range, for example wherein the clinical test result data is abnormally high, abnormally low, or invalid or erroneous.

In some examples, the at least one additional clinical test is a different clinical test to the first clinical test associated with the obtained clinical test data. For example, a first clinical test may test a blood sample for the presence of a specific pathogen A; where the result is negative, at least one additional clinical test may be required to identify the presence of a different pathogen, for example testing for the presence of specific pathogen B. However, in other examples, the at least one additional clinical test may be the same clinical test to the first clinical test. For example, in response to a determination that the clinical test result data is invalid or erroneous, the additional clinical test requested may comprise a repeat test of the first clinical test. However, in other examples, the additional clinical test may comprise a different type of clinical test to the first clinical test.

In some examples, the type of additional clinical test suggested by the method and/or automatically requested may be determined based on a pre-determined decision tree, wherein the method progresses through the decision tree based on the prior clinical test result data, for example based on the determination as to whether the data falls within the pre-determined range, to determine at least one type of additional clinical test. In other examples, the type(s) of additional clinical test requested may be determined based on a machine learning, neural network, and/or artificial intelligence algorithm, based on the prior clinical test result data, for example the determination as to whether the data falls within the predetermined range.

In some examples, the method may further comprise obtaining clinical information, wherein the clinical information relates to the patient corresponding to the sample and identification data. For example, the clinical information may relate to at least one of symptomatic presentation of the patient, medical history, and/or family medical history.

The clinical information may be received from manual input by a clinician, or retrieved from an API or remote server. In some examples, the type of additional clinical test suggested and/or automatically requested may be determined based on a pre-determined decision tree, wherein the method progresses through the decision tree based on the prior clinical test result data and the clinical information. In other examples, the type(s) of additional clinical test requested may be determined based on a machine learning, neural network, and/or artificial intelligence algorithm, based on the prior clinical test result data and the obtained clinical information.

In some examples, the method further comprises obtaining additional clinical test result data, wherein the additional clinical test result data relates to the requested additional clinical test.

In some examples, the method further comprises sending a signal to a remote server to store the clinical test result data and/or additional clinical test result data, for example using cloud computing. The stored clinical test result data, and/or additional clinical test result data, may be stored with the corresponding identification data, for example such that the clinical test result data is associated with the patient's medical records. The clinical test result data and/or additional clinical test result data may be stored as part of an EMR. This may be advantageous to ensure that the patient record is kept up to date at all times.

In some examples, the clinical test result data, and/or additional clinical test result data, is obtained from a laboratory information system. However, in other examples, the clinical test result data may be obtained from another third-party system, including directly from the laboratory.

In some examples, the method may further comprise sending a signal to alert a user that the clinical test result data and/or additional clinical test result data has been received. In some examples, the method may comprises sending a signal to display the clinical test result data and/or additional clinical test result data on a display, for example for viewing by a user, for example a clinician. The display may, additionally or instead, be configured to display the determination as to whether the clinical test result data and/or additional clinical test result data falls within the pre-determined range.

In some examples, the method may further comprise sending a signal to alert a user based on the determination as to whether the clinical test result data falls within the predetermined range, or above or below a predetermined threshold, for example in the event that the clinical test result data does not fall within the pre-determined range. This may be advantageous to escalate abnormal and/or concerning test results and reduce the time required for clinicians to manually check for specific test orders and results. The alert may comprise at least one of (i) a sound alert, or (ii) a visual alert, for example a flashing light or flashing display. In some examples, the alert may comprise a combination of a sound alert and a visual alert. This may be advantageous to escalate abnormal and/or concerning test results and reduce the time required for clinicians to manually check for specific test orders and results. Furthermore, this escalation may reduce lead times if further urgent action is required in response to the test results, for example but not limited to requesting additional clinical tests, or escalating the patient's treatment, for example administering urgent medication, or placing the patient under increased observation or into intensive care.

In some examples, sending the signal to alert a user may comprise sending at least one signal from a set of predetermined signals, wherein the set of predetermined signals comprises at least two of (i) a signal to alert the user that the clinical test data is abnormally low compared to the pre-determined range; (ii) a signal to alert the user that the clinical test data is abnormally high compared to the predetermined range; and (iii) a signal to alert the user that the clinical test data is invalid and/or unprocessed, for example due to error.

In some examples, the signal to alert the user that the clinical test data is abnormally low compared to the pre-determined range may cause a first visual alert and/or a first sound alert; the signal to alert the user that the clinical test data is abnormally high compared to the pre-determined range may cause a second visual alert and/or a second sound alert; and the signal to alert the user that the clinical test data is invalid and/or unprocessed may cause a third visual alert and/or a third sound alert. Preferably, the first, second, and third visual alerts and sound alerts are different to one another. However, the skilled person will understand these are merely examples and different visual alerts, sound alerts, and combinations of visual and sounds alerts may be used.

In some examples, the method may further comprise requesting an indication that the alert has been received and/or acknowledged by a medical professional. For example, the method may request at least one of a signature, name, initials, or personal identifier of a medical professional be input, for example via a computer system interface/graphical user interface, to acknowledge that the alert has been received. In some examples, the alert may be continually escalated until such an acknowledgement is received. Escalation may comprise but is not limited to at least one of: increasing the volume of the sound alert, increasing the frequency of the visual and/or sound alerts, sending a signal to alert another user device, for example such as a clinician pager device, sending a signal to alert another user.

In some examples, the method may further comprise sending at least a portion of the clinical test result data and/or additional clinical test result data to a remote device, for example the patient's remote device for viewing. Additionally, or instead, the method may further comprise sending at least a portion of the clinical test result data and/or additional clinical test result data to an API or webpage accessible by the patient.

As described above, the method comprises sending a signal to request at least one additional clinical test be performed on the sample corresponding to the identification data in the event that the clinical test result data does not fall within the pre-determined range. However, the skilled person will understand that in other examples, the signal to request at least one additional clinical test is sent by the processor based on the outcome that the clinical test result data does fall within the pre-determined range, or based on the outcome that the clinical test result data is above a pre-determined threshold, or below a pre-determined threshold.

In a second aspect of the invention, there is provided a computer-implemented method for escalating abnormal clinical test results, the method comprising obtaining identification data and obtaining clinical test result data, wherein the clinical test result data relates to a sample corresponding to the identification data. The method then comprises determining whether the clinical test result data falls above or below a predetermined threshold. In some examples, determining whether the clinical test result data falls above or below a predetermined threshold comprises determining whether the clinical test result data falls within a pre-determined range. Based on the determination, for example in the event that the clinical test result data does not fall within the pre-determined range, the method comprises sending a signal to alert a user, wherein sending the signal comprises sending at least one signal from a set of predetermined signals, wherein the set of predetermined signals comprises at least (i) a signal to alert the user that the clinical test data is abnormally low compared to the pre-determined range, and (ii) a signal to alert the user that the clinical test data is abnormally high compared to the predetermined range. This may be advantageous to escalate abnormal and/or concerning test results and reduce the time required for clinicians to manually check for specific test orders and results. Furthermore, this escalation may reduce lead times if further urgent action is required in response to the test results, for example but not limited to requesting additional clinical tests, or escalating the patient's treatment, for example administering urgent medication, or placing the patient under increased observation or into intensive care.

In some examples, the set of predetermined signals may further comprise a signal to alert the user that the clinical test data is invalid and/or unprocessed, for example due to error. In some examples, in response to a determination that the clinical test result data is invalid or erroneous, a request for a repeat test of the first clinical test may be sent. The request for the repeat test may be sent in relation to the same or a different sample.

In some examples, the alert may comprise a combination of a sound alert and a visual alert, for example a flashing light or flashing display. However, the skilled person will understand that in other examples, the alert may comprise only one of (i) a sound alert or (ii) a visual alert.

In some examples, the signal to alert the user that the clinical test data is abnormally low compared to the pre-determined range may cause a first visual alert and/or a first sound alert; the signal to alert the user that the clinical test data is abnormally high compared to the pre-determined range may cause a second visual alert and/or a second sound alert; and the signal to alert the user that the clinical test data is invalid and/or unprocessed may cause a third visual alert and/or a third sound alert. Preferably, the first, second, and third visual alerts and sound alerts are different to one another. However, the skilled person will understand these are merely examples and different visual alerts, sound alerts, and combinations of visual and sounds alerts may be used.

In some examples, the method may further comprise receiving a signal to indicate that the alert has been registered/acknowledged by a clinician or other user. In some examples, the signal may further comprise a user identifier, for example a name, initials, personal identifier, or signature, to indicate who has acknowledged the alert.

In some examples, in the event that no signal is received to indicate that the alert has been registered or acknowledged after a first time period, the method may comprise escalating the alert. Escalation may comprise but is not limited to at least one of: increasing the volume of the sound alert, increasing the frequency of the visual and/or sound alerts, sending a signal to alert another user device, for example such as a clinician pager device, sending a signal to alert another user.

In a third aspect of the invention there is provided a clinical test data management system comprising a processor configured to perform the method of any of the first aspect and/or the second aspect of the present disclosure.

In a fourth aspect of the present invention there is provided a computer reprogram product comprising program instructions configured to program a programmable device to perform the method of any of the first aspect and/or the second aspect of the present disclosure.

In a fifth aspect of the invention there is provided clinical test data management system comprising a processor, a Laboratory Information Management System (LIMS or LIS), and a sample storage facility. The processor is configured to obtain a request to perform a clinical test on a sample; communicate with the sample storage facility to request a sample be retrieved for clinical testing from a plurality of samples; communicate with the LIMS to request a clinical test be performed on the sample; obtain, from the LIMS, clinical test result data corresponding to the sample; and send a signal to alert a user in response to obtaining the clinical test result data.

In some examples, the processor may be further configured to determine whether the clinical test result data falls within a pre-determined range, and send the signal to alert a user based on the determination as to whether the clinical test result data falls within a pre-determined range. Additionally or instead, the processor may be configured to determine whether the clinical test result data falls above or below a pre-determined threshold, and send the signal to alert a user based on the determination as to whether the clinical test result data falls above or below the pre-determined threshold.

The processor may be further configured to send a signal to the LIMS to request an additional clinical test be performed on the sample corresponding to the identification data based on the obtained clinical test result data. In some examples, the processor may be configured to send the signal to the LIMS to request the additional clinical test be performed on the sample based on the determination as to whether the clinical test result data falls within a pre-determined range

In some examples the sample storage facility comprises an automatic retrieval system. In these examples, the processor may be configured to communicate with the sample storage facility to request the sample be automatically retrieved for clinical testing from a plurality of samples. In some examples, the sample storage facility automatic retrieval system comprises a robotic retrieval system, and wherein the processor is configured to communicate to the sample storage facility such that a sample is retrieved by the robotic retrieval system based on the identification data.

The system may further comprise a computer system interface (or graphical user interface), wherein the computer system interface may be configured to receive input data from a user and send the input data to the processor, for example wherein the input data comprises a request to perform a clinical test. In some examples, the input data may include at least one type of clinical test and a patient/sample identifier, for example a patient number and/or sample number.

The system may further comprise a memory, wherein the memory is configured to store the clinical test result data and/or additional clinical test result data. In some examples, the clinical test result data and/or additional clinical test result data may be stored as an EMR.

In some examples, the LIMS may comprise an interface wherein the processor is configured to send the signal to request a clinical test be performed on the sample to the LIMS interface.

In some examples, the system may further comprise at least one laboratory testing machine. Each laboratory testing machine is configured to receive a sample, for example from the sample storage facility; receive a signal to request a clinical test be performed on the sample; perform the requested clinical test to generate clinical test result data; and send a signal comprising the clinical test result data. In some examples, the LIMS is configured to send a signal to request the clinical test be performed on the sample to the at least one laboratory testing machine.

The system may additionally comprise a display, wherein the display is configured to display the clinical test result data and/or additional clinical test result data. The display may be further configured to display an alert, for example a visual alert, based on a determination as to whether the clinical test result data falls within the pre-determined range.

The system may also comprise an audio emitting device configured to emit a sound alert based on a determination as to whether the clinical test result data falls within the pre-determined range.

In some examples, the system may further comprise a communication module, wherein the communication module is configured to send signals from the processor to other systems or devices. The communication module may be configured for wireless communication, for example using Bluetooth, Wi-Fi, 3G, 4G, or 5G. In some examples, the system may utilise peer-to-peer communication between devices, such as WebRTC. In some examples, the communication module may communicate using an Internet of Things (IOT) gateway. In some examples, the communication module may communicate with a cloud network platform.

In some examples, the system may further comprise an audio emitting device, wherein the audio emitting device is configured to emit a sound alert based on the determination as to whether the clinical test result data falls within the predetermined range, or above or below the predetermined threshold.

In some examples, the processor may be part of the LIS or LIMS system, or vice versa.

### DRAWINGS

Embodiments of the disclosure will now be described, by way of example only, with reference to the accompanying drawings, in which:

FIG. 1 shows an example system of the present invention.

FIG. 2 shows a schematic of an example method, for example for use with the example system of FIG. 1.

FIGS. 3A and 3B show example schematics illustrating an example signalling architecture, for example for use with the system of FIG. 1 and method of FIG. 2.

FIG. 4 shows a schematic of an example method, for example for use with the example system of FIG. 1, or FIGS. 3A and 3B.

FIG. 5 shows an example decision tree for requesting additional clinical tests, for example for use with the methods of FIG. 2 or 4.

### SPECIFIC DESCRIPTION

Embodiments of the claims relate to a clinical test data management system and method, in particular for requesting additional clinical tests for a patient and escalating abnormal clinical test results.

It will be appreciated from the discussion above that the embodiments shown in the Figures are merely exemplary, and include features which may be generalised, removed, or replaced as described herein and as set out in the claims.

The steps, connections, and processing of information and signals illustrated in the figures, including, but not limited to, any block and flow diagrams and message sequence charts, may typically be performed in the same or in a different serial or parallel ordering and/or by different components and/or processes, threads, etc., and/or over different connections and be combined with other functions in other examples and figures, unless this disables the embodiment or a sequence is explicitly or implicitly required (e.g., for a sequence of obtain the value and process said read value, the value must be obtained prior to processing it, although some of the associated processing may be performed prior to, concurrently with, and/or after the read operation).

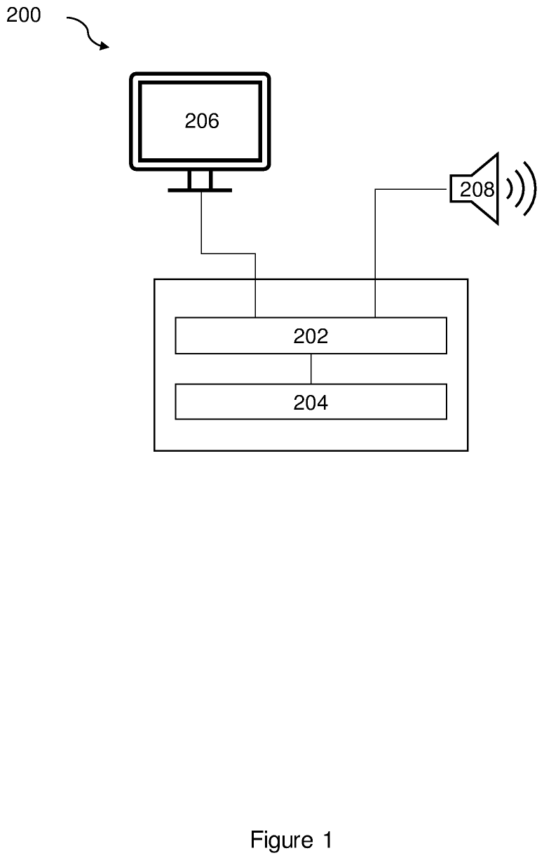

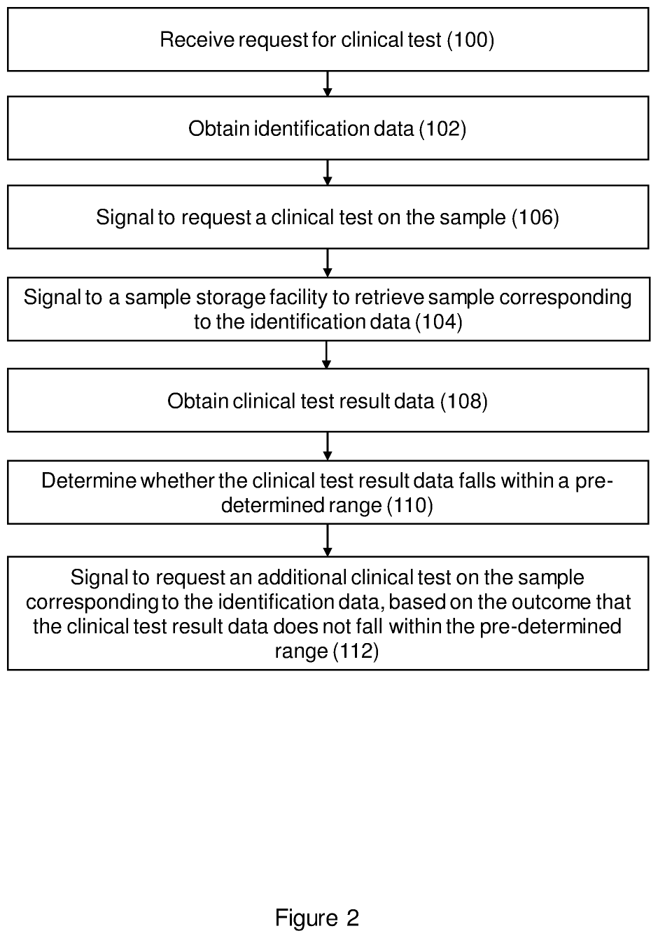

FIG. 1 shows an example system, for example for use with the method of FIG. 2. The system comprises a processor 202 and a memory 204. In this example, the system further comprises a display 206 and an audio emitting device 208, for example a speaker. The display 206 and audio emitting device 208 are coupled to the processor 202 such that the processor 202 is configured to signal to the coupled devices.

In some examples, the processor 202 may additionally comprise a Laboratory Information System (LIS) or Laboratory Information Management System (LIMS).

The processor is configured to perform the method of FIG. 2, discussed below.

The memory 204 is configured to store clinical test result data, including additional clinical test result data.

The display 206 is configured to display the clinical test result data and/or additional clinical test result data. The display 206 may also be configured to receive user input through a graphical user interface, for example wherein the user may request a clinical test, and optionally an additional clinical test. User input may be received by touch input, or input by a button, keyboard, mouse, or other suitable means. In this example, the display 206 and audio emitting device 208 are further configured to emit an alert based on a determination as to whether the clinical test result data falls within a predetermined range, for example in the event that the clinical test result data does not fall within a pre-determined range, according to the method of FIG. 2. For example, the display may be configured to display a visual alert, such as a flashing display and/or alert colour. The audio emitting device 208 may be configured to emit a sound alert. In this example, the example system is configured to emit both a visual alert and a sound alert; however, the skilled person will understand that in other examples, the system may be configured to emit either a visual alert or a sound alert.

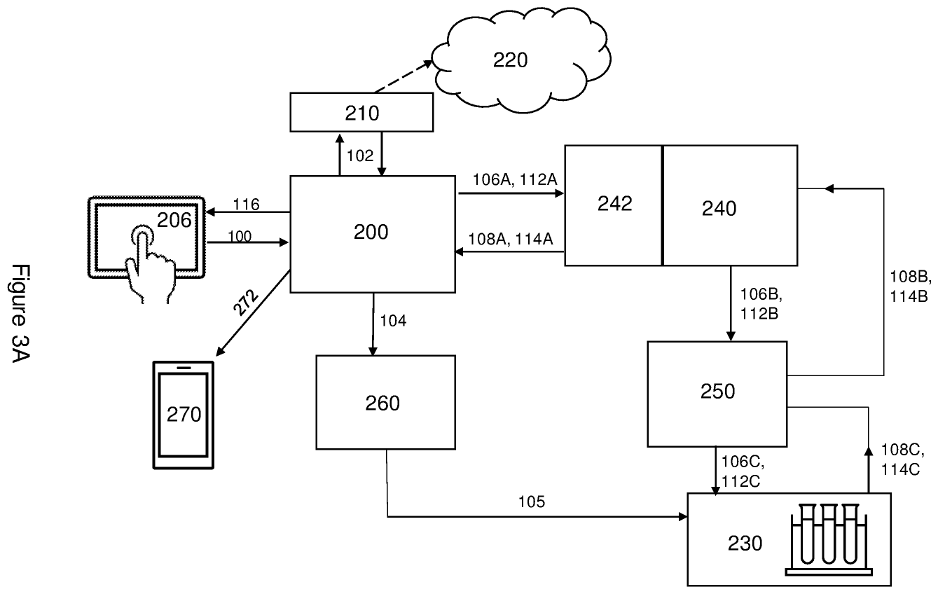

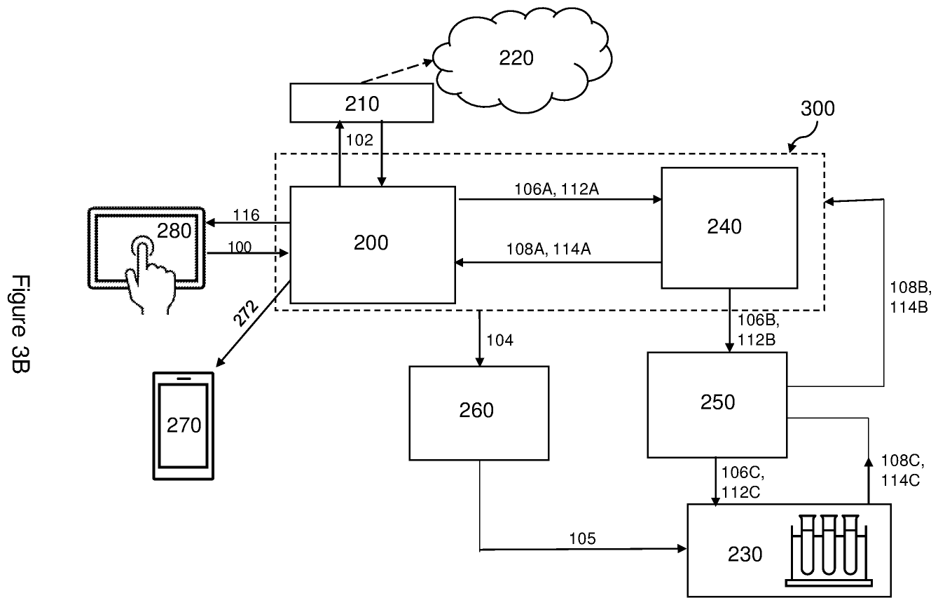

In use, the processor 202 performs the method of FIG. 2 wherein, firstly, the processor 202 receives a request for a clinical test to be performed on a sample (100). FIG. 3 shows an example schematic illustrating an example signalling architecture, for example for use with the system of FIG. 1 and method of FIG. 2. In the example shown in FIG. 3A, the system 200 of FIG. 2 is configured to communicate with one or more clinical data management systems and act as an intermediary therebetween. In particular, the system and method of FIGS. 1 and 2 requests additional specific clinical tests for a patient based on results from previous tests.

In the example shown in FIGS. 3A and 3B, the request for a clinical test (100) is received by the processor 202 in response to user input, for example wherein the request for a clinical test is input through a graphical user interface on the display 206 by a user, or otherwise wherein the request is input by a user using any other suitable device, for example a suitable ward device. The request may comprise patient identification data, for example but not limited to a name, patient number/identifier, or hospital number/identifier. The request may additionally comprise at least one type of clinical test to be performed, for example a blood test, however the skilled person will understand that any kind of clinical test may be requested.

The processor 202 then obtains additional identification data (102) based on the request, wherein the additional identification data corresponds to at least one patient sample, for example comprising sample number or identifier, wherein the sample number or identifier may be known to the sample storage facility and/or the laboratory. The sample identification data may be obtained based on the received patient identification data. Optionally, the additional identification data may comprise an EMR, patient data record and/or other clinical information corresponding to the patient. The other clinical information may relate to at least one of symptomatic presentation of the patient, medical history, and/or family medical history. In some examples, the additional identification data may be obtained from an API 210, for example wherein the processor 202 fetches the additional identification data from the API 210.

In the event that the additional identification data suggests that a plurality of samples exist corresponding the patient, the processor 202 may select at least one sample from the plurality of samples for the clinical test to be performed on. Selecting the at least one sample from a plurality of samples may be based on the requirements of the clinical test and/or the properties of the samples, for example but not limited to, based on age or “freshness” of a sample, type of sample, sample integrity, whether a sample may have been contaminated, etc. This may be advantageous to select the most suitable sample for the additional clinical test requested, for example, some types of clinical tests may require that the sample tested is not older than 24, 36, or 48 hours old. However, the skilled person will understand that in other examples, the sample to be tested may be chosen based on other sample properties, or by the clinician or laboratory staff.

Purely for illustrative purposes, the processor 202 may receive the request for a clinical test (100) in response to input from a clinician. In this purely illustrative example, the request may comprise the patient's name and date of birth, for example John Smith, 1 Jan. 1980, and the type of clinical test to be performed, for example a test for blood glucose levels. The processor 202 then obtains additional identification data (102) based on the request from an API. The additional identification data suggests that a plurality of samples exist corresponding the patient, John Smith, born 1 Jan. 1980: sample A001 (blood sample taken 72 hours ago), sample A002 (blood sample taken 24 hours ago), and sample B001 (urine sample taken 24 hours ago). The processor 202 selects one sample from the plurality of samples based on the requirements of the clinical test for blood glucose levels and/or the properties of the samples. In this case, the processor 202 selects sample A002 based on the factors that the clinical test for blood glucose levels must be performed on a blood sample and preferably should be performed on the freshest sample. However, the skilled person will understand that in other examples, the sample to be tested may be chosen based on other sample properties. In this example, the selection decision is performed by the processor 202, however in other examples, this selection decision may be performed on a remote device, such as a remote server, and the selected sample identification data may be obtained by the processor 202, for example as output by the API 220.

The processor 202 then sends a signal to request the clinical test be performed on the sample (106). In the example shown in FIG. 3A, the request is sent (106A) to the LIMS 240, for example via a LIMS interface API 242. In some examples, the system of FIG. 1 may further comprise the LIMS, for example as shown in FIG. 3B wherein the processor 202 and the LIMS 240 are coupled 300; however in other examples, the LIMS may be a third-party LIMS. In response to receiving the request, the LIMS 240 may then send the test request (106B) to a third-party system 250, which may send the test request (106C) to lab equipment 230 to perform the test. However, the skilled person will understand that in other examples, the LIMS 240 may then send the test request (106B) directly to lab equipment 230 to perform the test, or otherwise the processor 202 may send the signal to request the clinical test directly to the third-party system 250, or directly to the lab equipment 230.

The processor 202 also sends a signal to a sample storage facility 260 to retrieve the selected physical sample to be tested (104) based on the sample identification data, for example a sample number or identifier, in this case “sample A002”. The sample storage facility 260 may be an on-site facility, or a third-party facility. In the example shown in FIGS. 3A and 3B, the sample is retrieved from the sample storage facility 260 and sent (105) to the laboratory 230, for example wherein the sample is retrieved robotically using the sample identification data. For example, the sample may be robotically retrieved by correlating the identification data associated with the sample to a known location in the storage facility, for example position “X” in sample rack number “Y”. The identity of the retrieved sample may be verified based on the identification data, for example by scanning an identification tag on the sample, such as a barcode, QR code, RFID tag, or other suitable means.

Alternatively, the processor 202 may send a signal to a Laboratory Information Management System (LIMS) 240, and the LIMS 240 may signal to the sample storage facility 260 to retrieve the selected physical sample to be tested (104).

The lab equipment 230 may be part of the system, or third-party lab equipment. The lab equipment 230 may comprise at least one laboratory testing machine configured to receive the sample from the sample storage facility; receive a signal to request a clinical test be performed on the sample; perform the requested clinical test to generate clinical test result data; and send a signal comprising the clinical test result data. In some examples, the lab equipment may comprise a plurality of lab testing equipment machines, wherein each machine may be configured to perform different clinical tests. The plurality of lab testing machines may also be configured to receive different types of clinical samples.

After the clinical test has been performed, the processor 202 subsequently obtains the clinical test result data (108). In the example shown in FIG. 3A, the clinical test result data is obtained by the processor 202 (108A) from the Laboratory Information Management System (LIMS) 240, for example via the LIMS interface API 242. The LIMS 240 may obtain the clinical test result data (108B) from a third-party system 250, which may obtain the clinical test result data (108C) from lab equipment 230 or may be input manually by laboratory technicians. However, the skilled person will understand that in other examples, the LIMS 240 may obtain the clinical test result data directly from lab equipment 230, or the processor 202 may obtain the clinical test result data directly from the third-party system 250, or directly from the lab equipment 230.

The processor 202 then determines whether the clinical test result data falls within a pre-determined range (110). The pre-determined range may be determined based on the type of clinical test performed.

Optionally, in the event that the clinical test result data does not fall within the pre-determined range, the processor 202 signals to request at least one additional clinical test (also known as an add-on test) be performed on the sample corresponding to the identification data (112). This may be advantageous as additional clinical tests may be requested without having to repeat the retrieval of identification data corresponding to the patient. Preferably, the first clinical test and the at least one additional clinical tests are different to one another, however in other examples the first clinical test and the at least one additional clinical tests may be the same, for example wherein the first clinical test data is invalid or erroneous. Whilst in this example, the processor 202 sends a signal to request at least one additional clinical test be performed on the sample corresponding to the identification data in the event that the clinical test result data does not fall within the pre-determined range, the skilled person will understand that in other examples, the processor 202 may be configured, additionally or instead, to signal to request at least one additional clinical test is sent by the processor based on the outcome that the clinical test result data falls within the pre-determined range, or otherwise based on the outcome that the clinical test result data is above or below a pre-determined threshold.

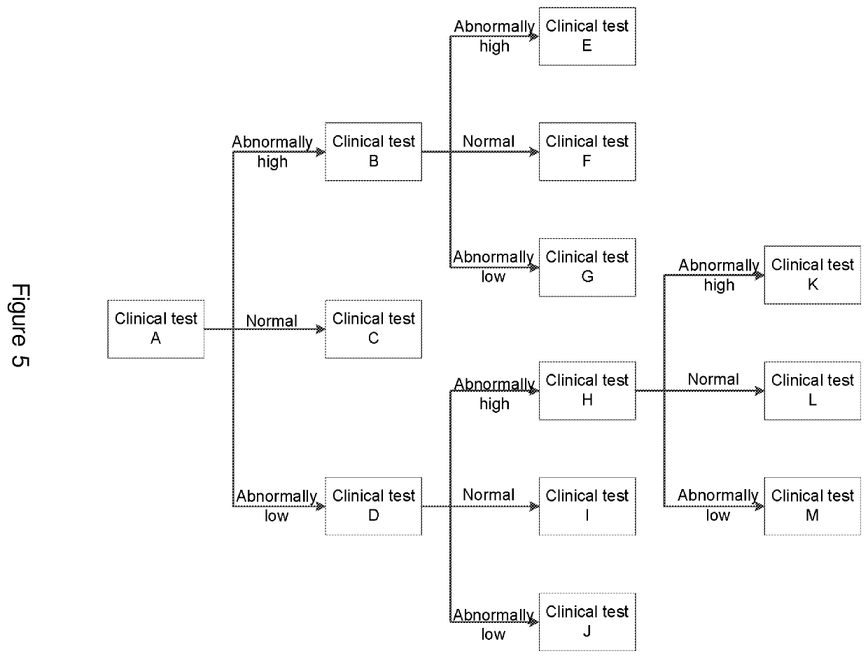

In some examples, the type(s) of additional clinical test requested may be determined based on a pre-determined decision tree, wherein the method progresses through the decision tree based on the prior clinical test result data, for example based on the determination as to whether the data falls within the pre-determined range or above or below a pre-determined threshold, to determine at least one type of additional clinical test. An example decision tree is shown in FIG. 5. In other examples, the type(s) of additional clinical test requested may be determined based on a machine learning (ML) and/or artificial intelligence (AI) algorithm, based on the prior clinical test result data, for example the determination as to whether the data falls within the pre-determined range. The type(s) of additional clinical test may additionally, or instead, be requested or confirmed by input from a user, for example from a medical professional.

Optionally, in response to obtaining the clinical test result data, the processor 202 sends a signal to alert the user that the result data has been received. In some examples, the processor 202 may send the signal to alert the user (116) based on the determination as to whether the clinical test result data falls within a predetermined range, or above or below a predetermined threshold, for example in the event that the first clinical test result data does not fall within the pre-determined range. Sending the signal may comprise sending at least one signal from a set of predetermined alert signals, wherein the set of predetermined signals comprises at least (i) a signal to alert the user that the clinical test data is abnormally low, for example compared to the pre-determined range or threshold, and (ii) a signal to alert the user that the clinical test data is abnormally high, for example compared to the predetermined range or threshold.

In this example, the signal to alert the user that the clinical test data is abnormally low causes the display 206 to display a flashing screen in a first colour, for example orange, and the audio emitting device 208 to emit a first sound alert, for example a continuous chirp sound. The signal to alert the user that the clinical test data is abnormally high causes the display 206 to display a flashing screen in a second colour, for example red, and the audio emitting device 208 to emit a second sound alert, for example continuous distinct sound.

Optionally, the set of predetermined signals may further comprise a signal to alert the user that the clinical test data is invalid and/or unprocessed, for example due to error. In this example, the signal to alert the user that the clinical test data is invalid and/or unprocessed causes the display 206 to display a flashing screen in a third colour, for example blue, and the audio emitting device 208 to emit a third sound alert. Preferably, the first, second, and third visual alerts and sound alerts are different to one another. However, the skilled person will understand these are merely examples and different visual alerts, colours, sound alerts, and combinations of visual and sounds alerts may be used.

Optionally, the processor may also request an indication that the alert has been received and/or acknowledged by a medical professional. For example, the processor may signal to request that at least one of a signature, name, initials, or personal identifier of a medical professional is input into a graphical user interface of the display 206 to acknowledge that the alert has been received. In some examples, the alert may be continually escalated until such an acknowledgement is received. Escalation may comprise but is not limited to at least one of: increasing the volume of the sound alert, increasing the frequency of the visual and/or sound alerts, sending a signal to alert another user device, for example such as a clinician pager device, sending a signal to alert another user.

In the example discussed above, the signal to request at least one additional clinical test is generated by the processor 202 based on the determination as to whether the clinical test result data falls within the pre-determined range. However, in other examples, the signal to request at least one additional clinical test may be sent by the processor 202 in response to user input, for example wherein a request for at least one additional clinical test is input through a graphical user interface on the display 206 by a user in response to the alert sent by the processor 202 to alert the user to the obtained the first clinical test result data (116). In some examples, the processor 202 may be configured to signal to the graphical user interface to suggest at least one additional clinical test, for example based on a pre-determined decision tree or ML or AI algorithm, and the user input is received in response to the at least one suggestion, for example confirming or declining the suggested additional clinical test.

In the example shown in FIG. 3A, the processor 202 sends the signal to request the at least one additional clinical test (112A) to the LIMS 240, for example via the LIMS interface API 242. The LIMS 240 may then send the additional test request (112B) to a third-party system 250, which may send the additional test request (112C) to lab equipment 230 to perform the test. However, the skilled person will understand that in other examples, the processor 202 may send the signal to request the at least one additional clinical test directly to the third-party system 250, or directly to the lab equipment 230. In the example shown in FIG. 3B, the LIMS 240 and the processor 202 are coupled 300; for example wherein the processor 202 comprises the LIMS 240.

After the at least one additional clinical test has been performed, the processor 202 subsequently obtains the additional clinical test result data (114). In the example shown in FIG. 3A, the additional clinical test result data is obtained by the processor 202 (114A) from the LIMS 240, for example via the LIMS interface API 242. The LIMS 240 may obtain the additional clinical test result data (114B) from a third-party system 250, which may obtain the additional clinical test result data (114C) from lab equipment 230 or may be input manually by laboratory technicians. However, the skilled person will understand that in other examples, the processor 202 may obtain the additional clinical test result data directly from the third-party system 250, or directly from the lab equipment 230.

Optionally, the processor 202 then determines whether the additional clinical test result data falls within a pre-determined range (110), or above/below a predetermined threshold. The pre-determined range or threshold may be determined based on the type of clinical test performed, thus the pre-determined range or threshold for the additional clinical test result data may be different to the pre-determined range or threshold for the first clinical test result data. Based on the determination, for example in the event that the clinical test result data does not fall within the pre-determined range, the processor 202 may then signal to request a second at least one additional clinical test on the sample corresponding to the identification data (112). However the skilled person will understand that the processor 202 may instead, or additionally, signal to request a second at least one additional clinical test on the sample corresponding to the identification data (112) in the event that the clinical test result data falls within the pre-determined range, or above or below a predetermined threshold. Preferably, the first additional clinical test(s) and the second additional clinical test(s) are different to one another, however in other examples the first additional clinical test(s) and the second additional clinical test(s) may be the same, for example wherein the first additional clinical test data is invalid or erroneous. The method may continue in this way, requesting additional clinical tests based on the outcome of prior clinical tests, until a diagnosis is reached, or the clinician is otherwise satisfied. In some examples, a diagnosis may be determined based on the previous clinical test result data for a patient, for example using a pre-determined decision tree or ML/AI algorithm.

The clinical test result data, including the additional clinical test result data, may be stored by the memory 204. Additionally, or instead, the processor 202 may send a signal to a remote server 220 to store the clinical test result data and/or additional clinical test result data, for example as cloud storage. Where the clinical test result data is stored by the memory 204 and a remote server 220, the remote server may provide back-up storage. The clinical test result data, including additional clinical test result data, may optionally be added to the patient's record using the identification data, for example the clinical test result data may be stored as an EMR.

In some examples, the system 200 may send at least a portion of the clinical test result data and/or additional clinical test result data (272) to a remote device 270, for example the patient's remote device, for example but not limited to a smartphone or personal computer. The patient then may be able to access at least a portion of their clinical test results, for example via the remote device or a webpage.

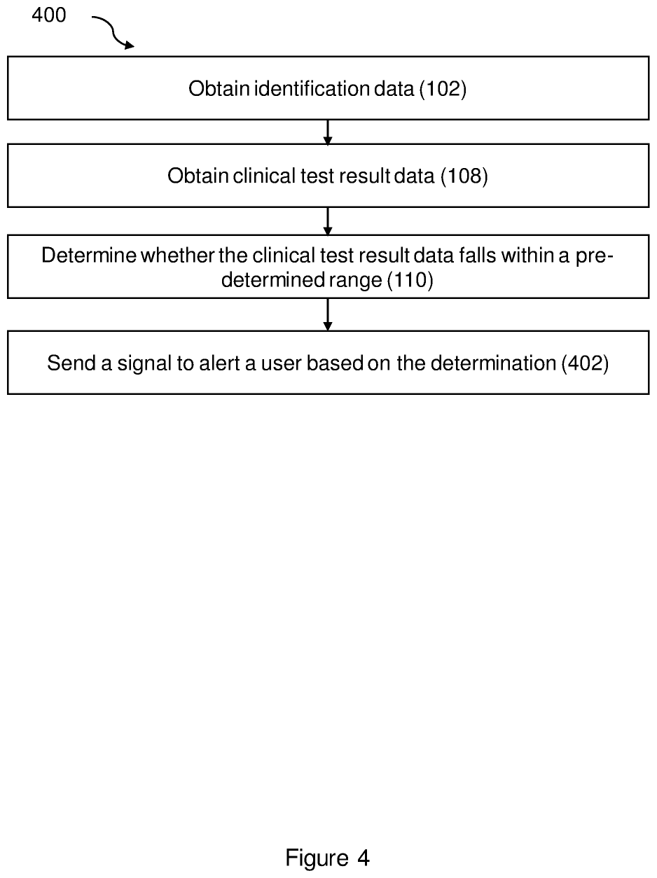

FIG. 4 shows an example method 400, for example for use with the device of FIG. 1 or the system of FIGS. 3A or 3B wherein the method comprises obtaining identification data (102) and obtaining clinical test result data (108), wherein the clinical test result data relates to a sample corresponding to the identification data. The method then comprises determining whether the clinical test result data falls within a pre-determined range (110).

In other examples, the method may additionally or instead comprise determining whether the clinical test result data falls above or below a pre-determined threshold. Based on the determination, for example in the event that the clinical test result data does not fall within the pre-determined range, the method comprises sending a signal to alert a user (402). This may be advantageous to escalate abnormal and/or concerning test results and reduce the time required for clinicians to manually check for specific test orders and results. The method 400 may be configured to be performed by the processor 202. Whilst the example described above discloses sending the signal to alert the user in the event that the clinical test result data does not fall within the pre-determined range, the skilled person will understand that the signal may instead, or additionally, be sent in the event that the clinical test result data falls within the pre-determined range, or above or below a predetermined threshold.

In this example, sending the signal to alert the user comprises sending at least one signal from a set of predetermined signals, wherein the set of predetermined signals comprises (i) a signal to alert the user that the clinical test data is abnormally low compared to the pre-determined range, (ii) a signal to alert the user that the clinical test data is abnormally high compared to the predetermined range, and (iii) a signal to alert the user that the clinical test data is invalid and/or unprocessed, for example due to error. This alert method may reduce lead times if further urgent action is required in response to the test results, for example but not limited to requesting additional clinical tests, or escalating the patient's treatment, for example administering urgent medication, or placing the patient under increased observation or into intensive care.

In response to a determination that the clinical test result data is invalid or erroneous, the processor 202 may be configured to send a request for a repeat test of the first clinical test. The request for the repeat test may be sent in relation to the same or a different sample but corresponding to the same patient.

In this example, the alert comprises a combination of (i) a sound alert emitted from audio emitting device 208, and (ii) a visual alert, for example a flashing light or flashing display 206. However, the skilled person will understand that in other examples, the alert may comprise only one of (i) a sound alert or (ii) a visual alert.

The signal to alert the user that the clinical test data is abnormally low may initiate a first visual alert and/or a first sound alert; the signal to alert the user that the clinical test data is abnormally high may initiate a second visual alert and/or a second sound alert; and the signal to alert the user that the clinical test data is invalid and/or unprocessed may initiate a third visual alert and/or a third sound alert; wherein the first, second, and third visual alerts and sound alerts are different to one another. However, the skilled person will understand these are merely examples and different visual alerts, sound alerts, and combinations of visual and sounds alerts may be used.

In this example, the processor 202 may receive a signal to indicate that the alert has been registered and/or acknowledged by a clinician or other medical professional user. In some examples, the signal may further comprise a user identifier, for example a name, initials, personal identifier, or signature, to indicate who has acknowledged the alert. This may be advantageous to record that the alert has been received and create a log of who has seen the alert and/or clinical test result data.

In the event that no signal is received to indicate that the alert has been registered or acknowledged after a first time period, the method may comprise escalating the alert. Escalation may comprise the processor 202 sending a signal to (i) increase the volume of the sound alert at the audio emitting device 208, (ii) increase the frequency of the visual and/or sound alerts at the display 206 or audio emitting device 208, (iii) sending a signal to alert another user device, for example such as a clinician pager device, and/or (iv) sending a signal to alert another user. The escalation cycle may continue to escalate the alert in time intervals until a signal is received to indicate that the alert has been registered or acknowledged by a medical professional.

The time intervals of escalation may be dependent on the test result data, for example highly concerning abnormal test result data may be escalated faster (i.e. at smaller time intervals) than less concerning abnormal or invalid test result data. This may be advantageous to prioritise urgent test result data being received by medical staff, and thus facilitating urgent care being actioned quickly for high-risk patients.

In the context of the present disclosure other examples and variations of the apparatus and methods described herein will be apparent to a person of skill in the art.

1-38 (canceled)

39. A computer-implemented method for requesting additional clinical tests for a patient, the method including:
obtaining identification data associated with the patient;
obtaining clinical test result data associated with the patient, wherein the clinical test result data relates to a biological sample of the patient corresponding to the identification data;
determining whether the clinical test result data falls above, within or below a pre-determined threshold;
sending a signal to request an additional clinical test be performed on the biological sample or a new biological sample corresponding to the identification data based on the determination as to whether the clinical test result data falls above, within or below the pre-determined threshold;
identifying whether at least one additional biological sample from a plurality of samples of the patient is suitable for the additional clinical test to be performed on, based on at least one of (i) requirements of the additional clinical test to be performed, and (ii) properties of each of the plurality of samples; and
selecting the at least one suitable biological sample from the plurality of samples corresponding to the identification data for the additional clinical test to be performed on, or, in the event that no suitable biological sample exists, sending an alert to request that a suitable biological sample is taken from the patient identified by the identification data.

40. The method of claim 39, wherein:
the step of determining whether the clinical test result data falls above, within or below the pre-determined threshold includes the step of determining whether the clinical test result data falls within or not within a pre-determined range; and
the step of sending the signal to request the additional clinical test is based on the determination as to whether the clinical test result data falls within or not within the pre-determined range.

41. The method of claim 40, wherein the signal is sent to request the additional clinical test in an event that the clinical test result data falls outside the pre-determined range.

42. The method of claim 39, wherein the step of sending the signal to request the additional clinical test be performed on the biological sample corresponding to the identification data further includes:
sending a second signal to a sample storage facility to request that the biological sample corresponding to the identification data be sent to a laboratory; and
sending a third signal to the laboratory to request the additional clinical test to be performed on the biological sample.

43. The method of claim 39 further comprising the step of receiving a request from a user for the additional clinical test and sending the signal to request the additional clinical test be performed on the biological sample in response to the request from the user.

44. The method of claim 39 further comprising the step of obtaining additional clinical test result data, wherein the additional clinical test result data relates to the requested additional clinical test.

45. The method of claim 39, wherein the step of obtaining the clinical test result data further includes:
obtaining a request from a user to perform the clinical test on the biological sample;
sending a signal to request the clinical test be performed on the biological sample corresponding to the identification data; and
obtaining the clinical test result data, wherein the clinical test result data relates to the requested clinical test.

46. The method of claim 39, wherein the identification data is obtained from an Application Programming Interface (API), and the clinical test result data is obtained from a laboratory information system.

47. The method of claim 39 further comprising the step of sending a signal to a remote server to store any one of or any combination of the clinical test result data and additional clinical test result data.

48. The method of claim 39 further comprising the step of:
sending a signal to alert a user in response to the clinical test result data being obtained, and in which sending the signal to alert the user further includes sending an alert to the user based on the determination as to whether the clinical test result data falls within a pre-determined range.

49. The method of claim 48, wherein the signal is at least one signal from a set of predetermined signals, wherein the set of predetermined signals comprising at least two of selected from the group consisting of a signal to alert the user that the clinical test result data is abnormally low compared to the pre-determined range, a signal to alert the user that the clinical test result data is abnormally high compared to the pre-determined range, and a signal to alert the user that the clinical test result data is invalid and/or unprocessed.

50. A clinical test data management system including:
a computer system including a processor and a memory;
a Laboratory Information Management System (LIMS); and
a biological sample storage facility including an automatic retrieval system;
in which program instructions executable by the processor cause the computer system to:
obtain a request to perform a clinical test on a biological sample;
communicate with the biological sample storage facility to request the biological sample be automatically retrieved for clinical testing from a plurality of biological samples;
communicate with the LIMS to request the clinical test to be performed on the biological sample;
obtain clinical test result data corresponding to the biological sample from the LIMS; and
send a signal to alert a user in response to obtaining the clinical test result data.

51. The system of claim 50, wherein the program instructions further cause the computer system to:
determine whether the clinical test result data falls within a pre-determined range, and
send the signal to alert the user based on the determination as to whether the clinical test result data falls within the pre-determined range.

52. The system of claim 50, wherein the program instructions further cause the computer system to send a LIMS signal to the LIMS to request an additional clinical test be performed on the biological sample corresponding to the identification data based on the obtained clinical test result data.

53. The system of claim 52, wherein the LIMS signal is sent to the LIMS to request the additional clinical test be performed on the biological sample based on the determination as to whether the clinical test result data falls within a pre-determined range or above a pre-determined threshold.

54. The system of claim 50, wherein the automatic retrieval system comprises a robotic retrieval system, and wherein the program instructions further cause the computer system to communicate to the biological sample storage facility such that the biological sample is retrieved by the robotic retrieval system based on the identification data.

55. The system of claim 50 further comprising a graphical user interface, wherein the graphical user interface is configured to receive input data from the user or a different user, and send the input data to the processor, wherein the input data comprising the request to perform the clinical test.

56. The system of claim 50, wherein the LIMS includes an interface, and wherein the program instructions further cause the computer system to send the signal to request the clinical test be performed on the biological sample to the LIMS interface.

57. The system of claim 50 further comprising at least one laboratory testing machine, in which the at least one laboratory testing machine is configured to:
receive the biological sample from the biological sample storage facility;
receive the signal to request the clinical test to be performed on the biological sample;
perform the requested clinical test to generate clinical test result data; and
send a response signal including the clinical test result data.

58. The system of claim 50 further comprising:
a display, wherein the display is configured to display any one of or any combination of the clinical test result data and additional clinical test result data, and wherein the display is further configured to display an alert based on the determination as to whether the clinical test result data falls within a pre-determined range; and
an audio emitting device configured to emit a sound alert based on the determination as to whether the clinical test result data falls within the pre-determined range.
